(19) United States
(12) Patent Application Publication
(10) Pub. No.: US 2009/0196790 A1
(43) Pub. Date: Aug. 6, 2009

(54) APPARATUS AND PROCEDURE FOR THE CLEANING OF CONTAINERS SUCH AS BOTTLES IN A BOTTLE FILLING PLANT OR CONTAINERS IN A CONTAINER FILLING PLANT

(76) Inventor: Daryoush SANGI, Hamburg (DE)

Correspondence Address: NILS H. LJUNGMAN & ASSOCIATES, P. O. BOX 130, GREENSBURG, PA, 15601-0130, US

(21) Appl. No.: 12/365,493

(22) Filed: Feb. 4, 2009

Related U.S. Application Data
(63) Continuation-in-part of application No. PCT/EP2007/006807, filed on Aug. 1, 2007.

(30) Foreign Application Priority Data
Aug. 5, 2006 (DE) 10 2006 036 763.4

(51) Int. Cl. A61L 2/18; A61L 2/04

(52) U.S. Cl. 422/28; 422/38; 422/302; 422/291

(57) ABSTRACT

An apparatus and procedure for the cleaning of containers such as bottles in a bottle filling plant or containers in a container filling plant. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/EP2007/006807, filed on Aug. 1, 2007, which claims priority from Federal Republic of Germany Patent Application No. 10 2006 036 763.4, filed on Aug. 5, 2006. International Patent Application No. PCT/EP2007/006807 was pending as of the filing date of this application. The United States was an elected state in International Patent Application No. PCT/EP2007/006807.

### BACKGROUND

1. Technical Field

The present application relates to an apparatus and procedure for the cleaning of containers such as bottles in a bottle filling plant or containers in a container filling plant.

2. Background Information

Background information is for informational purposes only and does not necessarily admit that subsequently mentioned information and publications are prior art.

Containers, for example beverage bottles, cans, medicament or medicine bottles, or medicament or medicine cans, must or should often be sterilized prior to filling so as to prevent, restrict, and/or minimize the material to be filled from spoiling or going bad. In the cooperation with the aseptic treatment installations or aseptic treatment stations and the filling installations or filling stations, filling of the containers can occur thus under clean room or clean space conditions by means of which a subsequent sterilization, e.g. by heating of the container together with the container contents, can be avoided, restricted, and/or minimized, which is desirable under certain circumstances as pertains to the quality and/or the taste of the filling material or the bottled material.

For this purpose in known installations, a liquid sterilization medium, a gaseous sterilization medium, or a steam sterilization medium is passed into the interior of the containers where a sterilization reaction takes place. Subsequently, as a function of the used medium, the container can be rinsed, for example, with sterile water.

It is possible that due to pertaining requirements, a sterilization of the interior of the container is not sufficient so as to achieve the desired degree of absence of germs that is often necessary or desired for a treatment stage, treatment phase, and/or during the filling of the container. In such an event, the exterior of the container must or should also be sterilized in a further treatment step, for instance, by spraying with a liquid sterilization medium, a gaseous sterilization medium, or a steam sterilization medium.

Such an approach is laborious and, accordingly, the used machines or equipment become more complicated and expensive. Also, every additional step in the process and the associated increase of the complexity of the installations lead to an increase of the sensitivity as to disruptions, the servicing intensity, and the like factors.

### OBJECT OR OBJECTS

An object or task of the present application is to realize the pertaining sterilization of containers in a very simple manner, but, nevertheless, with the extent or the degree of sterilization being sufficient for an aseptic treatment, or processing and filling.

### SUMMARY

The present application achieves this object by means of a device that is configured for the sterilization of containers in accordance with the features of a device for the sterilization of containers with a conduit-type arrangement or device for the introduction of a gaseous medium and/or steam sterilization medium through a container opening into the interior of a container. A guide-type arrangement is provided for the guiding of the sterilization medium that is exiting from the container to the outside wall of the container.

The present application relates to a device, as well as to a method or procedure for performing the sterilization of containers with the aid of a conduit-type arrangement or device that is configured for the introduction of a gaseous sterilization medium, and/or for the introduction of a steam sterilization medium through a container opening into the interior of a container.

In this way, a sterilization of the interior of the container and at least of parts of the container exterior is made possible in a single step. The second sterilization step for the outside or exterior surfaces of the container is, accordingly, rendered superfluous.

The guide-type arrangement or device contemplated in accordance with the present application, moreover, comprises in one possible embodiment a bell arrangement that is configured to surround the opening of the container. This bell is open in the direction of the bottom of the container and surrounds in coaxial manner the conduit-type arrangement or device which is configured, for instance, in the form of a pipe arrangement or in the form of a tube arrangement. The tube arrangement passes the sterilization medium through the interior of the container into the proximity of the bottom of the container, from where it is pressed in the direction of the opening of the container and thereby comes into operative or effective contact with the interior or inside wall surface of the container. Subsequently, a portion of the sterilization medium exits at the opening or mouth of the container and to the outside of the conduit-type arrangement and such portion is then diverted by the guide-type arrangement, so that the sterilization medium subsequently also comes into operative or effective contact with the outside wall of the bottle.

In addition, the bell can be tuned to or adapted to the used container type by comprising a shape that follows the exterior shape or contour of a container by way of a certain substantially constant distance, or by an increasing distance. Thereby, it can be sufficient according to the present application if the upper area, in one possible embodiment, the opening of the container and parts of the container neck are sterilized, because the remaining part of the bottle does not come into contact or does not touch the contents or the material in the container.

Alternatively, the guide-type arrangement can also be configured as a longitudinal tunnel. Also with such a configuration, the tunnel or tunnel structure is open in the direction of the container bottom and the bottles are moved together with the introduced conduit-type arrangement through the tunnel structure. The sterilization is otherwise conducted as described herein above, with the sterilization medium that is leaving through the opening of the container being guided by way of the tunnel walls to the exterior of the container.

In a continuous treatment process and/or filling process, the containers are taken up, for instance, in a rotary machine or a carousel-type arrangement on one side or end, and the containers are sterilized while the arrangement rotates. For this, the conduit-type arrangement is introduced through the opening of the container into the interior of the container until the conduit-type arrangement is, as described, with its opening positioned near the bottom of the container. During the positioning of the bottom of the container, or upon positioning of the conduit-type arrangement or device, the sterilization medium is guided or passed by this conduit-type into the interior of the container, so that the sterilization reaction can commence. After completed sterilization, the conduit-type arrangement or device, in the configuration together with the guide-type arrangement, is withdrawn through the opening of the container, so that the container is set free and can leave the sterilization device or arrangement.

In one possible embodiment of the present application, steam comprising H₂O₂ is used as the sterilization medium. The steam comprising H₂O₂ is introduced into the interior of the container and forms upon contacting the relatively cooler interior surfaces of the container a condensate film on the interior surfaces of the container, as well as on the surface areas of the outside wall of the container to which such steam comprising H₂O₂ has likewise been guided by the guide-type arrangement. After the application of the condensate film or condensation film, the H₂O₂ is activated, for instance, in an activation equipment through the supply of a certain amount of heat, for instance, by the use of hot air and/or steam. Upon the activation, a decay reaction of the H₂O₂ takes place during the course of which various free radicals are produced, which, for their part, react with the germs, the pollutants, and/or the impurities that are possibly present on the surfaces of the container and thus establish the sterilization mechanism. At the end of the decay reaction, there are at hand basically water as well as some remainders of the disintegration or decay reaction.

In at least one possible embodiment according to the present application, the apparatus disclosed in the present application could be used in an aseptic filling plant for the aseptic filling of containers or bottles.

These and further possible embodiments of the present application are evident according to the present application.

The above-discussed embodiments of the present invention will be described further herein below. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

Further possible embodiments of the present application are evident from the accompanying drawings, in which:

FIG. 1 shows a side view of the device in a first embodiment;

FIG. 2 shows a cross-section of the device in accordance with FIG. 1;

FIG. 3 shows a side elevational view, partially shown in cross-section, of an alternative arrangement of the present application;

FIG. 4 shows a side elevational view of another possible embodiment of the present application;

FIG. 4A shows an embodiment of the present application similar to that seen in FIG. 4;

FIG. 5 shows a top view of at least one possible embodiment of the present application;

FIG. 6 shows a top view of another embodiment, with the addition of an inlet and an outlet;

FIG. 7 shows an embodiment of the present application similar to that of FIGS. 3, 4, and 4A, with the addition of an annular ring;

FIG. 8 shows a side view of one embodiment of the present application used in a linear sterilization machine;

FIG. 8A shows a top view of the embodiment of FIG. 8; and

FIG. 9 shows schematically the main components of one possible embodiment example of a system for filling containers, specifically, a beverage bottling plant for filling bottles 130 with at least one liquid beverage, in accordance with at least one possible embodiment.

### DESCRIPTION OF EMBODIMENT OR EMBODIMENTS

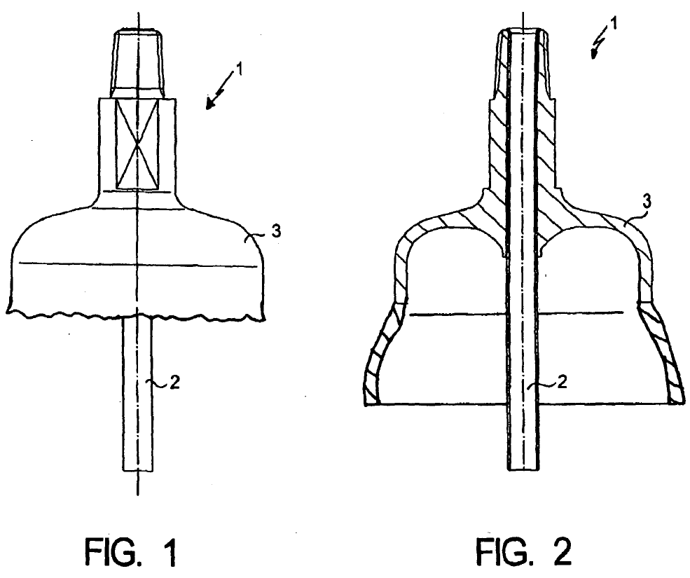

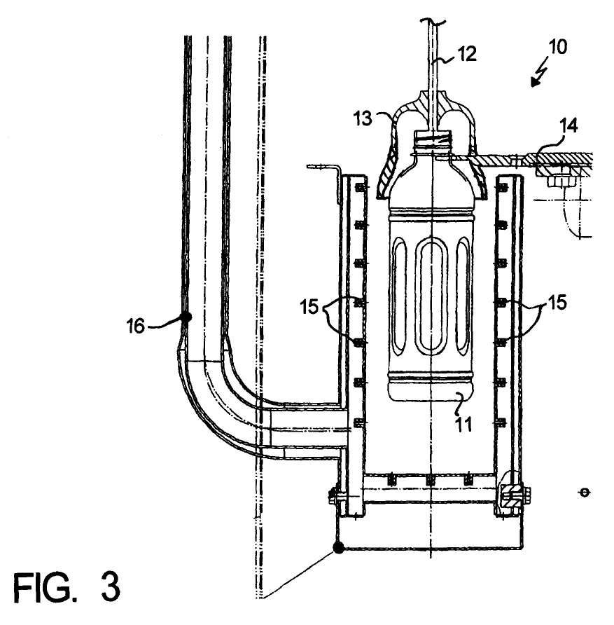

In general, in FIGS. 1 and 2 the reference numeral 1 identifies in detail a device configured for the sterilization of containers.

The device 1 comprises a pipe or tube 2 by means of which steam comprising H₂O₂ is introduced into the interior of a container, not shown, e.g., of a bottle or a can for a beverage.

In addition, the device 1 is configured with a bell 3 which coaxially surrounds the pipe 2.

For the sterilization stage, containers (not shown in detail in the drawings) are processed by a linear machine or arrangement or by a rotary machine, a carousel-type machine or arrangement. For the sterilization, the pipe 2 is introduced during the passage of the containers through the pertaining machine from above through the opening or mouth of the container. The length of the pipe 2 is by its design of such an extent so that it lies, upon the maximum insertion having been accomplished, in the area of the bottom of the container. According to an embodiment of the arrangement already discussed during the introduction, or, however, upon the pipe 2 having reached its lowermost position, steam comprising H₂O₂ is brought by means of the pipe 2 into the interior of the container.

The steam comprising H₂O₂ rises through the interior of the container and exits from the opening of the container while fully surrounding or enshrouding the pipe 2. During this phase the H₂O₂, on account of the temperature difference between the steam comprising H₂O₂ and the wall of the container, forms a condensation film. The H₂O₂ leaving from the opening of the container is diverted by the configuration of the bell 3, so that the steam comprising H₂O₂ does not just simply escape into the surroundings, but it is led onto the outside wall of the container in the region of the opening of the container, as well as in the region of the neck of the container. Also in these regions, the H₂O₂ forms a condensation film.

By way of the configuration of the bell 3, one can determine to what extent the condensation film that is formed can extend in the direction of the bottom of the container. The configuration of the film can be facilitated by adaptation of the shape of the bell 3 to the outer contour, or configuration of the container to be sterilized, with the bell 3 being shaped, for example, in such a way that the bell follows the outside contour of the container always or often by a constant distance, substantially constant distance, or by an increasing distance.

In at least one possible embodiment according to the present application, the H₂O₂ covers at least the mouth area, the neck ring area, or even the entire neck area of the container with a condensation film.

After a sufficient amount of steam comprising H₂O₂ has been introduced into the interior of the container, the supply of the medium is stopped.

When use is made of H₂O₂ as the sterilization medium, the H₂O₂ is activated through the supply of a certain amount of heat. For this activation, hot air or hot steam is blown into the interior or onto the exterior of the container, or the corresponding amount of heat is supplied by another known manner.

For instance, the hot air or the hot steam can be supplied by means of the pipe 2 with the bell 3 also serving to pass these media to the desired locations.

By way of the supplied amount of heat, a decay reaction is brought about during which the H₂O₂ disintegrates under substantially simultaneous or contemporaneous formation of free radicals. These free radicals react with, among other things, germs, other pollution, and/or impurities, and in this way sterilize the treated surfaces. At the end of the reaction, water and some decomposition products remain. If required and/or desired, the container can be rinsed, be dried, and/or be brought directly to a further treatment, processing, or to the filling stage.

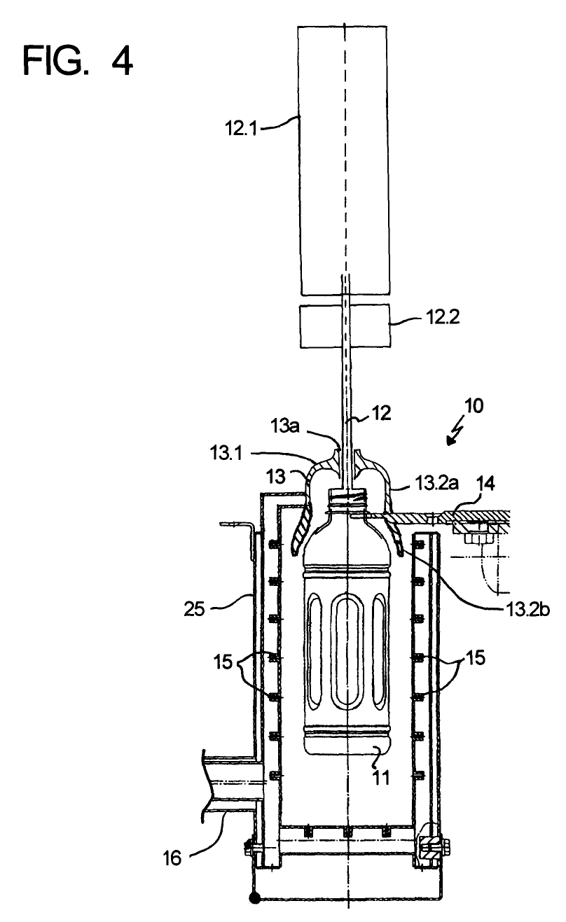

Another possible embodiment of the present application is shown in FIG. 3. The shown sterilization equipment 10 in accordance with the present application shows again a pipe 12 for the introduction of a steam sterilization means, such as H₂O₂, into the interior of a bottle or container 11 that is to be sterilized. The opening of the container 11, as well as the upper neck area are surrounded by a guide tunnel 13 which redirects the sterilization medium leaving from the container opening and passes it to the outside wall of the bottle 11. The bottle 11 is moved by a transport holder 14 through the stationary guide tunnel 13, whereby the pipe 12 is guided in synchronous or substantially synchronous manner with the bottle. For this operation, upon introduction of the bottle 11 into the sterilization arrangement 10, the pipe 12 is introduced from above into the entrance of the bottle 11 until the opening of the pipe 12 lies in the bottom region of the bottle 11. Upon completion of the sterilization the pipe 12 is pulled out of the container through the opening of the container. The sterilization equipment 10 that is shown in cross-section in FIG. 3 can be of a linear configuration, or can be of a rotary configuration or construction method, i.e., the bottles to be treated can be moved on a straight path or on a circular or curving path.

Also with this shown installation—upon use of H₂O₂—the condensation film that is the result of the condensation of the steam medium is activated through the supply of a pertaining amount of heat, so that, as already described, a sterilizing decay reaction is brought about. In the shown arrangement, this happens by contacting or by spraying the outside of the container with hot steam which exists through the illustrated nozzles 15 and which is introduced from a steam source, which is not shown in detail, by way of the conduit-type arrangement 16 into the machine. The activation is suitably done upon completion of the introduction of the sterilization medium, upon an effective condensation film could form in sufficient strength.

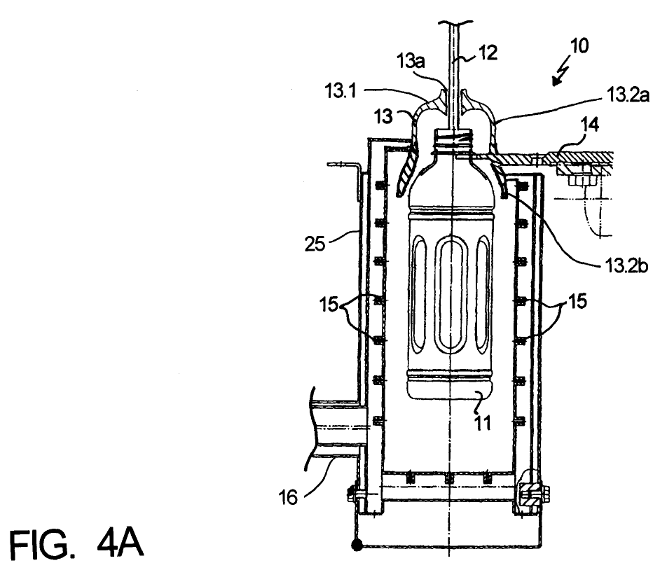

FIG. 4 shows one possible embodiment of a sterilization device 10 of the present application, which illustrates one possible configuration of a guide-tunnel 13, which is divided into multiple parts. The guide tunnel 13 also comprises a side 13.1, which faces outward and away from the rotary sterilization machine, and a side 13.2, which faces inward and toward the rotational axis of the rotary sterilization machine. The side 13.1 is fixed or attached to a hot steam or hot air dispensing device 25 of the conduit-type arrangement 16 adjacent to a plurality of nozzles 15.

The side 13.2 is also divided into two parts, a top portion 13.2a and a bottom portion 13.2b. In this embodiment illustrated in FIG. 4, both the top portion 13.2a and the bottom portion 13.2b are connected to the bottle holder 14, and therefore rotate with the bottle holder 14. The portions 13.1, 13.2a, and 13.2b together form the guide tunnel 13, even though side 13.1 is stationary and portions 13.2a and 13.2b rotate.

The configuration and/or shape of the guide tunnel 13 prevents, restricts, and/or minimizes the sterilization medium from evaporating or dissipating into the surroundings. The guide tunnel 13 of the embodiment seen in FIG. 4 comprises a slit 13a, which can be lined with a flexible material in order to restrict or minimize any sterilization medium from escaping through the slit 13a.

FIG. 4 shows a ring-shaped conduit 12.1 above the bottle 11 and pipe 12. The conduit 12.1 stores the cleaning or sterilization medium, which flows through the pipe 12 and is injected into the bottle 11, and the conduit 12.1 has a length which is substantially equal to the length of the bottle 11, in order to accommodate the vertical movement of the pipe 12. For example, when the pipe 12 is being moved out of the bottle 11, the pipe 12 moves up into the conduit 12.1 until it leaves the bottle 11. The conduit 12.1 is sufficiently long to allow the pipe 12 to move into the conduit 12.1 and still allow to liquid to flow into the pipe 12. Also seen in FIG. 4 is a motor 12.2. The motor 12.2 moves the pipe 12 in a vertical or substantially vertical direction, i.e. both into a bottle 11 and out of the conduit 12.1, and also out of a bottle 11 and into the conduit 12.1.

FIG. 4A shows a similar embodiment to that seen in FIG. 4. In this embodiment illustrated in FIG. 4A, the side 13.1 is connected to a portion of the stationary conduit-type arrangement 16. The top portion 13.2a is also similarly attached to the rotatable bottle or container holder 14. However, in this embodiment, the bottom portion 13.2b is connected to the hot steam or hot air dispensing device 25, and is therefore stationary.

In this embodiment, the portions 13.1 and 13.2b are stationary, and the top portion 13.2a rotates with the holder 14. Additionally, the slit 13a is open without any flexible material to surround the pipe 12, and the pipe is moved by the motor 12.2 into and out of the conduit 12.1, which is not shown in detail here.

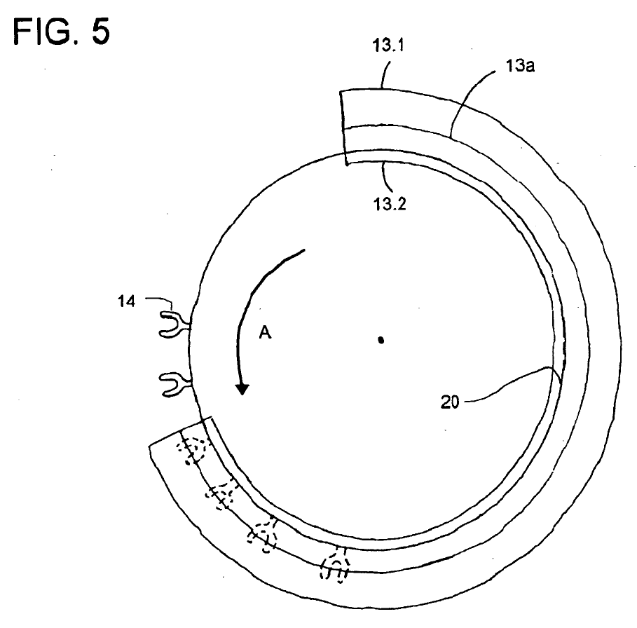

FIG. 5 is a top view of another possible embodiment of the present application applied to a rotary sterilization machine. In this embodiment, the guide-tunnel 13 comprises a bell shape or bell-like shape, which guides the sterilization medium, as the medium exits the bottle mouth or container mouth, around the mouth of the bottle or container as well as around the top of the bottle or container 11. The guide tunnel 13 comprises a slit 13a, which extends along the length of the guide tunnel 13. The slit 13a allows a plurality of pipes 12 to be inserted into bottles or containers 11 and also move along synchronously with the movement of a plurality of bottle holders 14 and the bottles 11 being held by the holders 14.

The guide tunnel 13 also comprises a side 13.1, which faces outward and away from the rotary sterilization machine, and a side 13.2, which faces inward and toward the rotational axis of the rotary sterilization machine. The guide tunnel 13 does not cover the entirety of the circumference of the sterilization machine, but rather a portion of the circumference. The length of the guide tunnel 13 can vary in differing embodiments.

The sterilization machine also comprises an annular ring or disc 20, on which a plurality of bottle holders or container holders 14 are mounted. The annular ring or disc 20 is configured to rotate about the rotary sterilization machine and transport bottles 11 into the guide tunnel 13 where the bottles 11 will be sterilized, and once the bottles 11 are sterilized, transport sterilized bottles 11 out of the guide tunnel 13 so the bottles 11 can then be filled or otherwise handled as desired. The annular ring or disc 20 is configured to rotate within the guide tunnel 13.

In the embodiment seen in FIG. 5, a bottle holder 14 holds a container 11. The motorized bottle holder 14 transports the container 11 into the guide tunnel 13. While the bottle holder 14 transports the container 11, a pipe 12 is inserted into the mouth of the container 11. A sterilization medium is passed into the interior of the container 11 through the pipe 12. In one possible embodiment, the sterilization medium comprises H₂O₂. The sterilization medium forms a film on the interior surfaces of the container 11. Sterilization medium is activated through a supply of an amount of heat T, through the application of hot air and/or steam. Upon activation of the sterilization medium, a decay reaction takes place, and as a result, the pollutants and germs are substantially removed and the container 11 is substantially sterilized. Any sterilization medium which leaves or exits the container 11 is directed onto the outside of the container 11 and also around the mouth area of the container 11 by the guide tunnel 13. The sterilization medium also forms a film on these exterior surfaces of the container 11. When heat, hot air, and/or steam is applied to the container 11 and activates the sterilization medium inside the container 11, the sterilization medium on the outside of the container 11 is also activated.

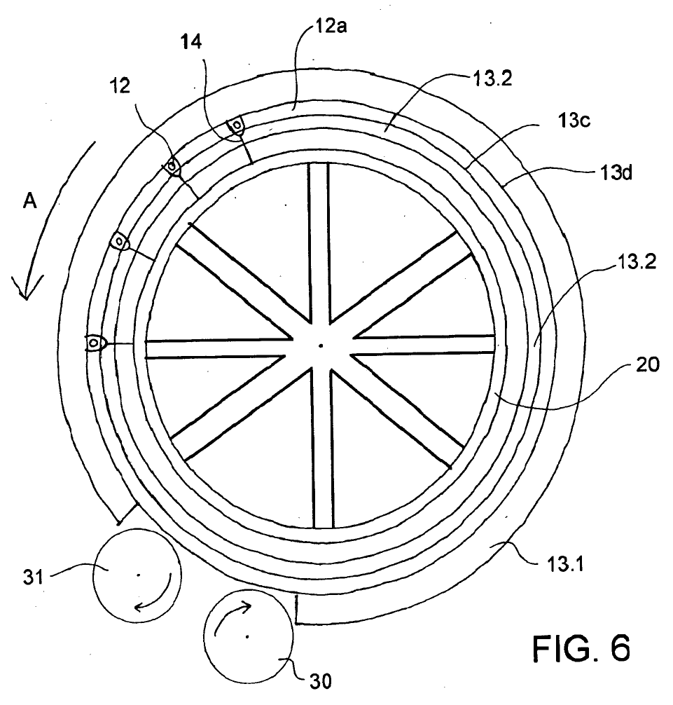

FIG. 6 shows a top view of one possible embodiment of the guide element 13 of the present application, coupled with a sterilization machine for the sterilization of bottles 11. In this one embodiment, the sterilization machine is configured as a rotary beverage bottle handling machine with a direction of rotation A. Inlet star wheel 30 feeds bottles 11 into the machine. The bottle holder 14 grabs the bottle 11 and transports the bottle 11 into the guiding element 13. The holder 14 is mounted on an annular ring 20. The bell shape of the guide tunnel 13 is formed by the two sides of the tunnel, 13.1 and 13.2 The side 13.2, which is closer to the rotational axis of the sterilization machine than the side 13.1, wraps around the circumference of the rotary machine and rotates with the bottle holders 14 as they transport containers 11 through the machine. The other side 13.1, also seen as the outer edge of the guide tunnel 13, partially covers the circumference of the rotary sterilization machine, which allows bottles 11 to be fed into the machine and also to be removed with the outlet star wheel 31.

Also seen in FIG. 6 is the plurality of pipes 12, which serve to inject sterilization medium into the bottles 11. The pipes 12 are spaced at predetermined intervals around the circumference of the rotary machine, and a pipe 12 corresponds for a bottle holder 14. The pipes 12 are configured to move vertically; for example, a pipe 12 will move in a downward direction to be inserted into a bottle 11, and the pipe 12 will move in an upward to exit the bottle 11. The pipes 12 rotate about the machine synchronously or substantially synchronously with the bottle holders 14 in the slit 13a. The slit 13a is formed as a gap between the sides 13.1 and 13.2. The side 13.1 comprises an edge 13d and the side 13.2 comprises the edge 13c, and together the edges 13c and 13d form the slit 13a.

The plurality of pipes are mounted on a ring 12a, which rotates about the machine. The ring 12a also serves to span the distances between the pipes 12 and at least partially cover the open space in the slit 13a. The ring 12a helps to restrict and/or minimize any sterilization medium from exiting the guide tunnel 13, and to promote that all or substantially all or most of the sterilization medium is activated in order to sterilize the bottles 11 in a sufficient manner.

Once a bottle 11 is fed into the machine by the inlet star wheel 30 and is transported into the guide tunnel 13, a pipe 12, which corresponds with the bottle holder 14 holding the bottle 11, is moved down into the bottle 11 through the bottle mouth. A sterilization medium is injected into the bottom the bottle 11. The sterilization medium rises in the bottle 11, and a portion exits the bottle 11 through the mouth. The guide tunnel 13 guides and directs the exiting sterilization medium onto the exterior of the bottle 11 around the mouth of the bottle 11. The sterilization medium is then activated with heat, and the interior of the bottle 11 as well at least a portion of the exterior surfaces of the bottle 11 are sterilized. The pipe 12 is lifted out of the bottle, and the bottle 11 leaves the sterilization machine through the outlet star wheel 31.

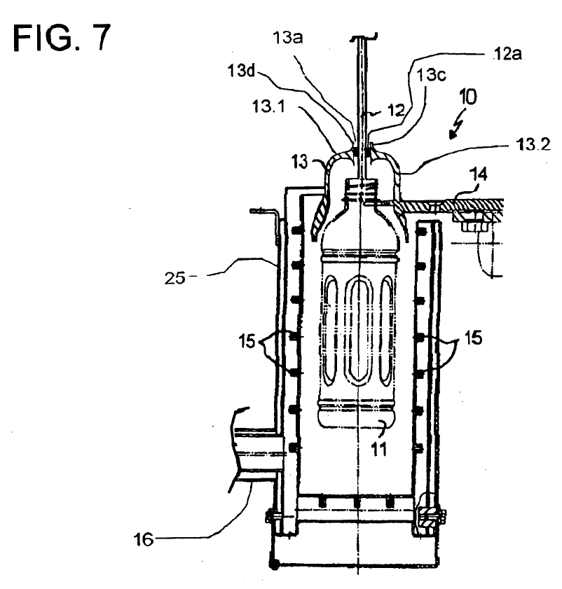

FIG. 7 shows another possible embodiment of the sterilization device according to the present application, similar to that seen in FIGS. 4 and 4A. In this one embodiment, the bottle holder 14 comprises the guide tunnel side 13.2. Also seen in FIG. 7 is the ring 12a, on which the pipe 12 is mounted. The ring 12a helps to prevent, restrict, and/or minimize any sterilization medium from exiting the guide tunnel 13 and to promote the sterilization of bottles 11.

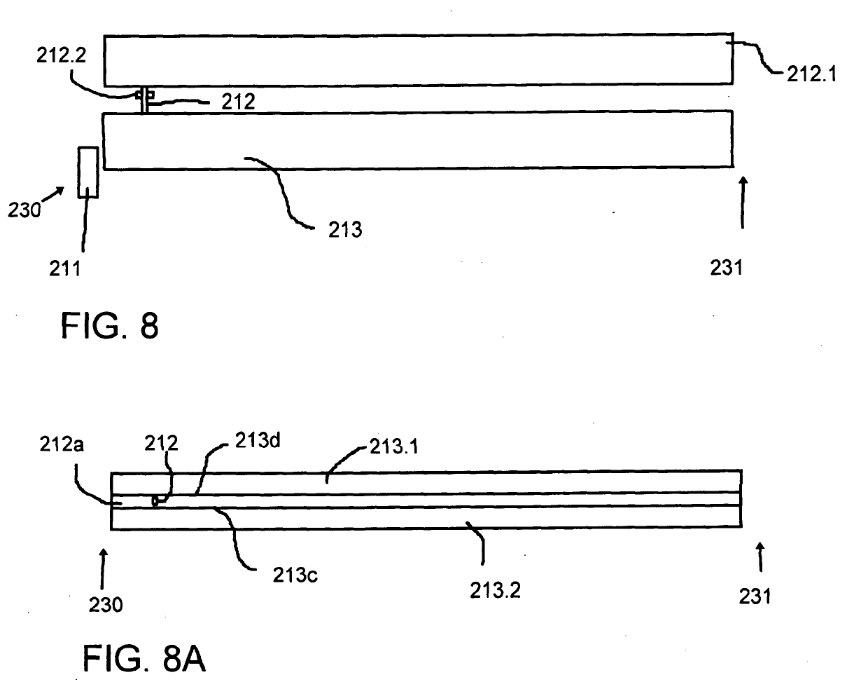

FIG. 8 shows one embodiment of the device of the present application in a linear sterilization machine. Bottles or containers 211 enter the linear sterilization through the input 230. As the bottle 211 enters the linear sterilization machine, the bottle 211 is surrounded be the guide tunnel arrangement 213. A pipe or conduit 212 is inserted into the bottle or container 211 by use of a motor 212.2, and a sterilization medium from the linear conduit 12.1, which stores the sterilization medium, is injected into the bottle 211. Some of the sterilization medium exits the bottle 211 through the bottle opening and flows onto the exterior surfaces of the bottle 211, for example around the opening or neck of the bottle 211. Heat is then applied to the bottle 211 to activate the sterilization medium, through means not seen in FIG. 8. The bottle 211 is then sterilized, and exits the machine through the outlet 231.

FIG. 8A shows a top view of the embodiment which is seen in FIG. 8. Additionally seen here is a ring 212a on which the pipe or conduit 212 is mounted. The edge 213d of the side 213.1 of the guide tunnel 213 abuts the ring 212a, and the edge 213c of the side 213.2 of the guide tunnel 213 abuts the ring 212a. A bottle or container 211 enters the linear sterilization machine through the input 230 and exits through the output 231.

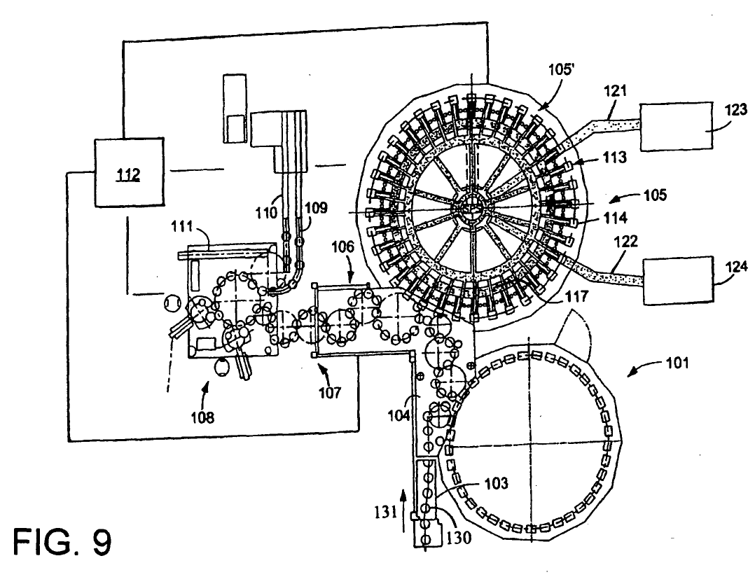

FIG. 9 shows schematically the main components of one possible embodiment example of a system for filling containers, specifically, a beverage bottling plant for filling bottles 130 with at least one liquid beverage, in accordance with at least one possible embodiment, in which system or plant could possibly be utilized at least one aspect, or several aspects, of the embodiments disclosed herein.

FIG. 9 shows a rinsing arrangement or rinsing station 101, to which the containers, namely bottles 130, are fed in the direction of travel as indicated by the arrow 131, by a first conveyer arrangement 103, which can be a linear conveyor or a combination of a linear conveyor and a starwheel. Downstream of the rinsing arrangement or rinsing station 101, in the direction of travel as indicated by the arrow 131, the rinsed bottles 130 are transported to a beverage filling machine 105 by a second conveyer arrangement 104 that is formed, for example, by one or more starwheels that introduce bottles 130 into the beverage filling machine 105.

The beverage filling machine 105 shown is of a revolving or rotary design, with a rotor 105′, which revolves around a central, vertical machine axis. The rotor 105′ is designed to receive and hold the bottles 130 for filling at a plurality of filling positions 113 located about the periphery of the rotor 105′. At each of the filling positions 103 is located a filling arrangement 114 having at least one filling device, element, apparatus, or valve. The filling arrangements 114 are designed to introduce a predetermined volume or amount of liquid beverage into the interior of the bottles 130 to a predetermined or desired level.

The filling arrangements 114 receive the liquid beverage material from a toroidal or annular vessel 117, in which a supply of liquid beverage material is stored under pressure by a gas. The toroidal vessel 117 is a component, for example, of the revolving rotor 105′. The toroidal vessel 117 can be connected by means of a rotary coupling or a coupling that permits rotation. The toroidal vessel 117 is also connected to at least one external reservoir or supply of liquid beverage material by a conduit or supply line. In the embodiment shown in FIG. 9, there are two external supply reservoirs 123 and 124, each of which is configured to store either the same liquid beverage product or different products. These reservoirs 123, 124 are connected to the toroidal or annular vessel 117 by corresponding supply lines, conduits, or arrangements 121 and 122. The external supply reservoirs 123, 124 could be in the form of simple storage tanks, or in the form of liquid beverage product mixers, in at least one possible embodiment.

As well as the more typical filling machines having one toroidal vessel, it is possible that in at least one possible embodiment there could be a second toroidal or annular vessel which contains a second product. In this case, each filling arrangement 114 could be connected by separate connections to each of the two toroidal vessels and have two individually-controllable fluid or control valves, so that in each bottle 130, the first product or the second product can be filled by means of an appropriate control of the filling product or fluid valves.

Downstream of the beverage filling machine 105, in the direction of travel of the bottles 130, there can be a beverage bottle closing arrangement or closing station 106 which closes or caps the bottles 130. The beverage bottle closing arrangement or closing station 106 can be connected by a third conveyer arrangement 107 to a beverage bottle labeling arrangement or labeling station 108. The third conveyor arrangement may be formed, for example, by a plurality of starwheels, or may also include a linear conveyor device.

In the illustrated embodiment, the beverage bottle labeling arrangement or labeling station 108 has at least one labeling unit, device, or module, for applying labels to bottles 130. In the embodiment shown, the labeling arrangement 108 is connected by a starwheel conveyer structure to three output conveyer arrangements: a first output conveyer arrangement 109, a second output conveyer arrangement 110, and a third output conveyer arrangement 111, all of which convey filled, closed, and labeled bottles 130 to different locations.

The first output conveyer arrangement 109, in the embodiment shown, is designed to convey bottles 130 that are filled with a first type of liquid beverage supplied by, for example, the supply reservoir 23. The second output conveyer arrangement 110, in the embodiment shown, is designed to convey bottles 130 that are filled with a second type of liquid beverage supplied by, for example, the supply reservoir 124. The third output conveyer arrangement 111, in the embodiment shown, is designed to convey incorrectly labeled bottles 130. To further explain, the labeling arrangement 108 can comprise at least one beverage bottle inspection or monitoring device that inspects or monitors the location of labels on the bottles 130 to determine if the labels have been correctly placed or aligned on the bottles 130. The third output conveyer arrangement 111 removes any bottles 130 which have been incorrectly labeled as determined by the inspecting device.

The beverage bottling plant can be controlled by a central control arrangement 112, which could be, for example, computerized control system that monitors and controls the operation of the various stations and mechanisms of the beverage bottling plant.

The present application is not limited to the preceding embodiment examples, but can be modified in varied manner without leaving the basic scope, spirit, or idea of the present application. Thus, in one possible embodiment, the precise or substantially precise or general configuration of the guide-type arrangement can be varied and is adaptable to accommodate the type of the container and the necessary and/or desired degree of sterilization. Also, the used materials and sterilization media can be modified to be suitably adapted to the application purpose.

With a device and a procedure for the sterilization of containers 11 with a conduit-type arrangement 2, 12 for the introduction of a gaseous medium and/or a steam sterilization medium through a container opening into the interior of a container 11, an embodiment-solution is to be provided with which containers 11 are sterilized in a very simple manner, and which containers, nevertheless, are sterilized to a degree that is sufficient for an aseptic treating or further processing and filling.

This is reached by the fact that a guide-type arrangement 3, 13 is provided for the guiding of the sterilization medium that is exiting from the container onto the outside wall of the container 11.

One feature or aspect of an embodiment is believed at the time of the filing of this patent application to possibly reside broadly in a device for the sterilization of containers 11 with a conduit-type arrangement, or device 2, 12 for the introduction of a gaseous medium, and/or steam sterilization medium through a container opening into the interior of a container 11, wherein a guide-type arrangement 3, 13 is provided for the guiding of the sterilization medium that is exiting from the container to the outside wall of the container 11.

Another feature or aspect of an embodiment is believed at the time of the filing of this patent application to possibly reside broadly in the device, wherein the guide-type arrangement 3, 13 that is configured for the guiding of the sterilization medium that is exiting from the container 11 onto the outside wall is configured to guide the sterilization medium in the region of at least one of: the area of the container opening, and/or the neck ring area, and/or the container neck, and/or the shoulder area.

Yet another feature or aspect of an embodiment is believed at the time of the filing of this patent application to possibly reside broadly in the device, wherein the guide-type arrangement 3, 13 comprises a bell which is configured to surround the opening of the container.

Still another feature or aspect of an embodiment is believed at the time of the filing of this patent application to possibly reside broadly in the device, wherein the guide-type arrangement 3, 13 comprises a tunnel structure that is configured to partially surround the opening of the container.

A further feature or aspect of an embodiment is believed at the time of the filing of this patent application to possibly reside broadly in the device, wherein the sterilization medium comprises H₂O₂.

Another feature or aspect of an embodiment is believed at the time of the filing of this patent application to possibly reside broadly in the device, wherein an activation equipment, or device is provided for the sterilization medium.

Yet another feature or aspect of an embodiment is believed at the time of the filing of this patent application to possibly reside broadly in the device, wherein the sterilization arrangement or device is configured in a linear construction, or in a rotary construction.

Still another feature or aspect of an embodiment is believed at the time of the filing of this patent application to possibly reside broadly in a method for the sterilization of containers with an arrangement, or device in accordance with the present application, wherein a gaseous medium, and/or steam sterilization medium is brought by means of the conduit-type arrangement into the interior of a container and the sterilization medium upon exiting through the opening of the container is guided by the guide-type arrangement onto the outside wall of the container.

A further feature or aspect of an embodiment is believed at the time of the filing of this patent application to possibly reside broadly in the method, wherein the sterilization medium is introduced at the bottom area of the container and rises up in the direction of the opening of the container.

One feature or aspect of an embodiment is believed at the time of the filing of this patent application to possibly reside broadly in a device for the sterilization of containers 11 with a conduit-type arrangement, or device 2, 12 for the introduction of a gaseous medium, and/or steam sterilization medium through a container opening into the interior of a container 11 and a guide-type arrangement, or device 3, 13 for the guiding of the sterilization medium that is exiting from the container onto the outside wall of the container 11, wherein the guide-type arrangement 3, 13 comprises a bell which is configured to surround the opening of the container.

Another feature or aspect of an embodiment is believed at the time of the filing of this patent application to possibly reside broadly in the device, wherein the guide-type arrangement 3, 13 is configured to provide for the guidance of the sterilization medium upon exiting from the container 11 onto the outside wall in the area of the container opening, and/or in the area of the neck of the container.

Yet another feature or aspect of an embodiment is believed at the time of the filing of this patent application to possibly reside broadly in the device, wherein the guide-type arrangement 3, 13 comprises an annular dome-shaped structure that is configured to surround the opening of the container.

Still another feature or aspect of an embodiment is believed at the time of the filing of this patent application to possibly reside broadly in the device, wherein the guide-type arrangement 3, 13 comprises a tunnel structure that is configured to partially surround the opening of the container.

A further feature or aspect of an embodiment is believed at the time of the filing of this patent application to possibly reside broadly in the device, wherein that the sterilization medium comprises H₂O₂.

Another feature or aspect of an embodiment is believed at the time of the filing of this patent application to possibly reside broadly in the device, wherein an activation equipment, or device is provided for the sterilization medium.

Yet another feature or aspect of an embodiment is believed at the time of the filing of this patent application to possibly reside broadly in the device, wherein the sterilization device is configured in a linear construction, or in a rotary construction.

Still another feature or aspect of an embodiment is believed at the time of the filing of this patent application to possibly reside broadly in a method for the sterilization of containers with a sterilization device according to the present application, wherein a gaseous medium, and/or a steam sterilization medium, which medium is configured to be activated, is brought by means of the conduit-type arrangement, or device into the interior of a container 11 and the sterilization medium upon exiting through the opening of the container is guided by the guide-type arrangement, or device 3, 13 onto the outside wall of the container 11 and is activated by an addition of a certain amount of heat.

A further feature or aspect of an embodiment is believed at the time of the filing of this patent application to possibly reside broadly in the method, wherein the sterilization medium is introduced at the bottom area of the container and rises in the direction of the opening of the container.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

The following patents, patent applications or patent publications, are hereby incorporated by reference as if set forth in their entirety herein.

All of the patents, patent applications or patent publications, which were cited in the International Search Report dated Nov. 9, 2007, and/or cited elsewhere are hereby incorporated by reference as if set forth in their entirety herein as follows: U.S. Pat. No. 4,511,538, having the title “APPARATUS FOR STERILIZING PACKAGING CONTAINERS,” published on Apr. 16, 1985; WO 79/01074, having the title “METHOD AND MEANS FOR APPLYING BACTERICIDE TO CONTAINER FOR STERILIZATION,” published on Dec. 13, 1979; and U.S. 2004/0208781, having the title “ARTICLE STERILIZING METHODS AND STERILIZING DEVICE,” published on Oct. 21, 2004.

All of the patents, patent applications or patent publications, which were cited in the German Office Action dated Apr. 4, 2007, and/or cited elsewhere are hereby incorporated by reference as if set forth in their entirety herein as follows: DE 38 09 855 A1, having the following English translation of the German title “METHOD FOR FILLING CONTAINERS WITH LIQUIDS AND IN ASEPTIC OR STERILE WAY, AND APPARATUS FOR CARRYING OUT THIS METHOD,” published on Feb. 9, 1989.

The patents, patent applications, and patent publication listed above, beginning on line 761 on page 37 in the paragraph with the phrase: “All of the patents, patent applications or patent publications, . . . ” and ending on line 779 on page 38 in the paragraph with the phrase: “ . . . published on Feb. 9, 1989” are herein incorporated by reference as if set forth in their entirety. The purpose of incorporating U.S. patents, Foreign patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. 10 2006 036 763.4, filed on Aug. 1, 2007, having inventor Daryoush SANGI, and DE-OS 10 2006 036 763.4 and DE-PS 10 2006 036 763.4, and International Application No. PCT/EP2007/006807, filed on Aug. 5, 2006, having WIPO Publication No. WO2008/017410 and inventor Daryoush SANGI, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the Foreign equivalent patent application PCT/EP2007/006807 and German Patent Application 10 2006 036 763.4 is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2007/006807 and DE 10 2006 036 763.4 from which this patent application claims priority which do not have to do with the correction of the translation in this patent application are not to be included in this patent application in the incorporation by reference.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

### AT LEAST PARTIAL NOMENCLATURE

1 Device for the sterilization of containers
2 Pipe, conduit-type arrangement, or device
3 Bell, guide-type arrangement, or device
10 Sterilization equipment
11 Container
12 Pipe
13 Guide tunnel
14 Transport holder
15 Nozzles
16 Conduit-type arrangement

What is claimed is:

1. A method for the cleaning of beverage bottles in a beverage bottling plant; said method comprising the steps of:
feeding a first beverage bottle into a beverage bottle cleaning machine;
inserting a pipe into said first beverage bottle, said pipe being configured to inject a cleaning medium into an opening of said first beverage bottle;
surrounding, substantially simultaneously with injecting, said opening of said first beverage bottle with a bell-shaped cleaning-medium-containing device, said bell-shaped cleaning-medium containing device comprising a bell-shaped cross-section and being configured to guide said cleaning medium onto at least a portion of the exterior surfaces of said first beverage bottle, upon said cleaning medium exiting said first beverage bottle through said opening;
injecting said cleaning medium into the bottom of said first beverage bottle with said pipe;
permitting rising of said injected cleaning medium in said first beverage bottle and overflowing a portion of said injected cleaning medium through said opening of said first beverage bottle;
containing said cleaning medium about at least a portion of the exterior surfaces of said first beverage bottle with said bell-shaped cleaning-medium-containing device, upon said cleaning medium exiting said first beverage bottle through said opening;
applying heat to said cleaning medium in said first beverage bottle and activating said cleaning medium in said first beverage bottle and on said at least a portion of said exterior surfaces of said first beverage bottle; and
cleaning the interior of said first beverage bottle and said at least a portion of said exterior surfaces of said first beverage bottle thereby.

2. Means for performing the method according to claim 1, said means comprising:
means for feeding a first beverage bottle into a beverage bottle cleaning machine;
means for inserting a pipe into a first beverage bottle, which pipe is configured to inject cleaning medium into an opening of a first beverage bottle;
means for surrounding, substantially simultaneously with injecting, an opening of a first beverage bottle with said means for surrounding, said means for surrounding comprising a bell-shaped cross-section and being configured to guide cleaning medium onto at least a portion of the exterior surfaces of a first beverage bottle, upon cleaning medium exiting a first beverage bottle through an opening;
means for injecting cleaning medium into a bottom of a first beverage bottle with a pipe;
said means for injecting cleaning medium into a bottom of a first beverage bottle comprises means for permitting rising of injected cleaning medium in a first beverage bottle and overflowing a portion of injected cleaning medium through an opening of a first beverage bottle;
means for containing cleaning medium about at least a portion of the exterior surfaces of a first beverage bottle with said means for surrounding, upon cleaning medium exiting a first beverage bottle through an opening of a first beverage bottle;
means for applying heat to cleaning medium in a first beverage bottle to activate cleaning medium in a first beverage bottle and on at least a portion of the exterior surfaces of a first beverage bottle to clean the interior of a first beverage bottle and at least a portion of the exterior surfaces of a first beverage bottle.

3. The method according to claim 1, wherein:
said cleaning medium further comprises a sterilization medium;
said at least a portion of the exterior surfaces of said first beverage bottle further comprises said opening and a neck of said first beverage bottle; and
said sterilization medium further comprises hydrogen peroxide.

4. The method according to claim 3, wherein said bell-shaped cleaning-medium-containing device:
further comprises an at least partially annular ring; and
is either configured as a part of a rotary beverage bottle sterilization machine or as a part of a linear beverage bottle sterilization machine.

5. Means for performing the method according to claim 4, said means comprising:
means for feeding a first beverage bottle into a beverage bottle cleaning machine;
means for inserting a pipe into a first beverage bottle, which pipe is configured to inject cleaning medium into an opening of a first beverage bottle;
means for surrounding, substantially simultaneously with injecting, an opening of a first beverage bottle with said means for surrounding, said means for surrounding comprising a bell-shaped cross-section and being configured to guide cleaning medium onto at least a portion of the exterior surfaces of a first beverage bottle, upon cleaning medium exiting a first beverage bottle through an opening;
means for injecting cleaning medium into a bottom of a first beverage bottle with a pipe;
said means for injecting cleaning medium into a bottom of a first beverage bottle comprises means for permitting rising of injected cleaning medium in a first beverage bottle and overflowing a portion of injected cleaning medium through an opening of a first beverage bottle;
means for containing cleaning medium about at least a portion of the exterior surfaces of a first beverage bottle with said means for surrounding, upon cleaning medium exiting a first beverage bottle through an opening of a first beverage bottle;
means for applying heat to cleaning medium in a first beverage bottle to activate cleaning medium in a first beverage bottle and on at least a portion of the exterior surfaces of a first beverage bottle to clean the interior of a first beverage bottle and at least a portion of the exterior surfaces of a first beverage bottle;
said means for surrounding further comprises an at least partially annular ring; and
said means for surrounding is either configured as a part of a rotary beverage bottle sterilization machine or as a part of a linear beverage bottle sterilization machine.

6. The method according to claim 3, wherein said bell-shaped cleaning-medium-containing device is configured:
to form a tunnel arrangement; and
either as a part of a rotary beverage bottle sterilization machine or as a part of a linear beverage bottle sterilization machine.

7. Means for performing the method according to claim 6, said means comprising:
means for feeding a first beverage bottle into a beverage bottle cleaning machine;
means for inserting a pipe into a first beverage bottle, which pipe is configured to inject cleaning medium into an opening of a first beverage bottle;
means for surrounding, substantially simultaneously with injecting, an opening of a first beverage bottle with said means for surrounding, said means for surrounding comprising a bell-shaped cross-section and being configured to guide cleaning medium onto at least a portion of the exterior surfaces of a first beverage bottle, upon cleaning medium exiting a first beverage bottle through an opening;
means for injecting cleaning medium into a bottom of a first beverage bottle with a pipe;
said means for injecting cleaning medium into a bottom of a first beverage bottle comprises means for permitting rising of injected cleaning medium in a first beverage bottle and overflowing a portion of injected cleaning medium through an opening of a first beverage bottle;
means for containing cleaning medium about at least a portion of the exterior surfaces of a first beverage bottle with said means for surrounding, upon cleaning medium exiting a first beverage bottle through an opening of a first beverage bottle;
means for applying heat to cleaning medium in a first beverage bottle to activate cleaning medium in a first beverage bottle and on at least a portion of the exterior surfaces of a first beverage bottle to clean the interior of a first beverage bottle and at least a portion of the exterior surfaces of a first beverage bottle;
said means for surrounding is configured to form a tunnel arrangement; and
said means for surrounding is either configured as a part of a rotary beverage bottle sterilization machine or as a part of a linear beverage bottle sterilization machine.

8. A method for the sterilization of containers with a sterilization-medium-containing arrangement; said method comprising the steps of:
feeding containers into a container sterilization machine;
inserting a conduit into said containers, said conduit being configured to inject a sterilization medium into the openings of said containers;
surrounding said openings of said containers with a sterilization-medium-containing arrangement, said sterilization-medium-containing arrangement being configured to guide said sterilization medium onto at least a portion of the exterior surfaces of said containers, upon said sterilization medium exiting said containers through said openings;
injecting said sterilization medium into the bottoms of said containers with said conduit;
permitting rising of said injected sterilization medium in said containers and overflowing a portion of said injected sterilization medium through said openings of said containers;
containing said sterilization medium about at least a portion of the exterior surfaces of said containers with said sterilization-medium-containing arrangement, upon said sterilization medium exiting said containers through said openings; and
applying heat to said sterilization medium in containers and activating said sterilization medium in said containers and on said at least a portion of said exterior surfaces of said containers.

9. Means for performing the method according to claim 8, said means comprising:
means for feeding containers into a container sterilization machine;
means for inserting a conduit into containers, which conduit is configured to inject sterilization medium into openings of containers;
means for surrounding openings of containers with said means for surrounding, said means for surrounding being configured to guide sterilization medium onto at least a portion of the exterior surfaces of containers, upon sterilization medium exiting containers through the openings;
means for injecting sterilization medium into bottoms of containers with a conduit;
said means for injecting sterilization medium comprises means for permitting rising of injected sterilization medium in containers and overflowing a portion of injected sterilization medium through openings of containers;
means for containing sterilization medium about at least a portion of the exterior surfaces of containers with said means for surrounding, upon sterilization medium exiting containers through openings; and
means for applying heat to sterilization medium in containers to activate sterilization medium in containers and on at least a portion of the exterior surfaces of containers.

10. The method according to claim 8, wherein said sterilization-medium-containing arrangement comprises a bell-shaped cross-section.

11. The method according to claim 10, wherein said at least a portion of the exterior surfaces of said containers further comprises said openings and the necks of said containers.

12. The method according to claim 11, wherein said sterilization medium further comprises hydrogen peroxide.

13. The method according to claim 12, wherein said sterilization-medium-containing arrangement further comprises an at least partially annular ring.

14. The method according to claim 13, wherein said sterilization-medium-containing arrangement is either configured as a part of a rotary container sterilization machine or as a part of a linear container sterilization machine.

15. Means for performing the method according to claim 14, said means comprising:
means for feeding containers into a container sterilization machine;
means for inserting a conduit into containers, which conduit is configured to inject sterilization medium into openings of containers;
means for surrounding openings of containers with said means for surrounding, said means for surrounding comprising a bell-shaped cross-section and being configured to guide sterilization medium onto at least a portion of the exterior surfaces of containers, upon sterilization medium exiting containers through the openings;
means for injecting sterilization medium into bottoms of containers with a conduit;
said means for injecting sterilization medium comprises means for permitting rising of injected sterilization medium in containers and overflowing a portion of injected sterilization medium through openings of containers;
means for containing sterilization medium about at least a portion of the exterior surfaces of containers with said means for surrounding, upon sterilization medium exiting containers through openings;
means for applying heat to sterilization medium in containers to activate sterilization medium in containers and on at least a portion of the exterior surfaces of containers;
said means for surrounding further comprises an at least partially annular ring; and
said means for surrounding is either configured as a part of a rotary container sterilization machine or as a part of a linear container sterilization machine.

16. An arrangement configured to perform the method according to claim 14, said arrangement comprising:
a feeding arrangement configured and disposed to feed containers into a container sterilization machine;
an insertion arrangement configured and disposed to insert a conduit into containers, which conduit is configured to inject sterilization medium into openings of containers;
a surrounding arrangement configured and disposed to surround openings of containers with said surrounding arrangement, said surrounding arrangement comprising a bell-shaped cross-section and being configured to guide sterilization medium onto at least a portion of the exterior surfaces of containers, upon sterilization medium exiting containers through the openings;
an injection arrangement configured and disposed to inject sterilization medium into bottoms of containers with a conduit;
said injection arrangement comprises a permitting arrangement configured and disposed to permit rising of injected sterilization medium in containers and overflowing a portion of injected sterilization medium through openings of containers;
a containing arrangement configured and disposed to contain sterilization medium about at least a portion of the exterior surfaces of containers with said surrounding arrangement, upon sterilization medium exiting containers through openings;
a heat application arrangement configured and disposed to apply heat to sterilization medium in containers to activate sterilization medium in containers and on at least a portion of the exterior surfaces of containers;
said surrounding arrangement further comprises an at least partially annular ring; and
said surrounding arrangement is either configured as a part of a rotary container sterilization machine or as a part of a linear container sterilization machine.

17. The method according to claim 12, wherein said sterilization-medium-containing arrangement is configured to form a tunnel arrangement.

18. The method according to claim 17, wherein said sterilization-medium-containing arrangement is either configured as a part of a rotary container sterilization machine or as a part of a linear container sterilization machine.

19. Means for performing the method according to claim 18, said means comprising:
means for feeding containers into a container sterilization machine;
means for inserting a conduit into containers, which conduit is configured to inject sterilization medium into openings of containers;
means for surrounding openings of containers with said means for surrounding, said means for surrounding comprising a bell-shaped cross-section and being configured to guide sterilization medium onto at least a portion of the exterior surfaces of containers, upon sterilization medium exiting containers through the openings;
means for injecting sterilization medium into bottoms of containers with a conduit;
said means for injecting sterilization medium comprises means for permitting rising of injected sterilization medium in containers and overflowing a portion of injected sterilization medium through openings of containers;
means for containing sterilization medium about at least a portion of the exterior surfaces of containers with said means for surrounding, upon sterilization medium exiting containers through openings;
means for applying heat to sterilization medium in containers to activate sterilization medium in containers and on at least a portion of the exterior surfaces of containers;
said means for surrounding is configured to form a tunnel arrangement; and
said means for surrounding is either configured as a part of a rotary container sterilization machine or as a part of a linear container sterilization machine.

20. An arrangement configured to perform the method according to claim 19, said arrangement comprising:
a feeding arrangement configured and disposed to feed containers into a container sterilization machine;
an insertion arrangement configured and disposed to insert a conduit into containers, which conduit is configured to inject sterilization medium into openings of containers;
a surrounding arrangement being configured and disposed to surround openings of containers with said surrounding arrangement, said surrounding arrangement comprising a bell-shaped cross-section and being configured to guide sterilization medium onto at least a portion of the exterior surfaces of containers, upon sterilization medium exiting containers through the openings;
an injection arrangement configured and disposed to inject sterilization medium into bottoms of containers with a conduit;
said injection arrangement comprises a permitting arrangement configured and disposed to permit rising of injected sterilization medium in containers and overflowing a portion of injected sterilization medium through openings of containers;
a containing arrangement configured and disposed to contain sterilization medium about at least a portion of the exterior surfaces of containers with said surrounding arrangement, upon sterilization medium exiting containers through openings;
a heat application arrangements configured and disposed to apply heat to sterilization medium in containers to activate sterilization medium in containers and on at least a portion of the exterior surfaces of containers;
said surrounding arrangement is configured to form a tunnel arrangement; and
said surrounding arrangement is either configured as a part of a rotary container sterilization machine or as a part of a linear container sterilization machine.
